(19) United States
(12) Patent Application Publication
(10) Pub. No.: US 2023/0285492 A1
(43) Pub. Date: Sep. 14, 2023

(54) PHYTOTHERAPEUTIC COMPOSITION

(71) Applicants: Tomas Lluis Manent, Barcelona (ES); Eric François Faucillon, Vannes (FR)

(72) Inventors: Tomas Lluis Manent, Barcelona (ES); Eric François Faucillon, Vannes (FR)

(21) Appl. No.: 18/019,671

(22) PCT Filed: Aug. 13, 2021
(86) PCT No.: PCT/IB2021/057464

§ 371 Date: Feb. 3, 2023

(30) Foreign Application Priority Data
Aug. 21, 2020 (GB) 2013130.6
Jan. 19, 2021 (GB) 2100710.9

(51) Int. Cl. A61K 36/81; A61K 36/8962; A61K 36/9068; A61K 36/9066; A61K 9/00; A61P 31/14

(52) U.S. Cl. CPC A61K 36/81; A61K 36/8962; A61K 36/9068; A61K 36/9066; A61K 9/0056; A61K 9/0073; A61P 31/14

(57) ABSTRACT

A phytotherapeutic composition, for the treatment of Covid-19 and other viral infections, comprising nicotine-containing parts of a species of the genus Nicotiana, optionally in combination with one or more of the species Allium sativum, Zingiber officinale and Curcuma longa, in the form of a liquid nasal, liquid oral, or liquid ocular formulation.

This invention relates to a phytotherapeutic composition and in particular to a phytotherapeutic antiviral agent containing tobacco.

A viral infection that is a global concern is Covid-19, which is an infectious disease caused by the coronavirus SARS-CoV-2. This virus and disease have caused a serious pandemic, with hundreds of thousands of deaths globally. There is a need for an effective antiviral to treat Covid-19.

The present invention tackles this problem by the use of a plant materials, either alone or in combination, in specific nasal, oral or ocular forms of administration. Phytotherapeutic remedies are well known in the treatment of a range of diseases. Alongside pharmaceutical drugs, medicinal plant materials make an important contribution to health care. Indeed, many phytotherapeutic remedies have proved to be more active than their isolated constituent compounds because of synergistic mechanisms between the compounds within a plant or between different plants.

In particular, the present invention relates to antiviral phytotherapeutic compositions comprising nicotine-containing parts of tobacco (Nicotiana sp) in specific nasal, oral or ocular formulations. The formulations of the whole plant tobacco, optionally supplemented by other plant species as enhancers or modulators, provide an effective treatment for viral infections including Covid-19.

Some literature has suggested the use of nicotine as an antiviral.

Changeux et al (C R Biol. 2020 Jun. 5; 343(1):33-39) suggests that the nicotinic acetylcholine receptor (nAChR) may play a key role in the pathophysiology of SARS-CoV-2 infection; and suggests that nicotine may be a potential preventive agent against Covid-19 infection.

Farsalinos et al, Toxicology Reports, Volume 7, 2020, Pages 658-663, also hypothesizes that Covid-19 may be a disease of the nicotinic cholinergic system and that nicotine could maintain or restore the function of the cholinergic anti-inflammatory system.

US patent application 2020/179367 relates to a method of treating a coronavirus comprising administering isomyosmine, which is a constituent of tobacco.

Nasal sprays of nicotine are known, which are used to relieve withdrawal symptoms and reduce the cravings for nicotine as an aid to stop smoking.

None of those documents disclose, or suggest, that tobacco can be used as a whole plant containing a combination of alkaloids that act synergistically to enhance antiviral activity. The use of tobacco, which is a composition of various specific components specific to tobacco acting in synergy, is very different from the use of nicotine alone

Chinese patent 103750539 describes a tobacco nasal spray that includes tobacco extract and Chinese herbal medicine extracts. However, the document relates to anti-inflammatory and antibacterial activity and does not describe nasal sprays of tobacco either alone, or in combination with any of the other plant species of the present invention.

WO 2009/110775 describes a process for the production of medicinal tobacco oil from tobacco seeds. Oral and nasal liquid formulations are described with inter alia antiviral activity. However, the process is specific to wastes from tobacco fields, i.e. tobacco seeds. There is no suggestion to use tobacco leaves. Furthermore, the formulations are specifically stated to have an absence of nicotine; and there is no suggestion that the formulations are useful to treat coronaviruses.

Bui et al, ACS Omega 2020, 5, 8312-8320, describes compounds found in garlic essential oil having an inhibitory effect on the host receptor ACE2 protein. The paper suggests that “garlic essential oil is a valuable natural antivirus source, which contributes to preventing the invasion of coronavirus into the human body.” However, there is no suggestion in the document to combine garlic, (Allium sativum) with tobacco or the other plant species of this invention.

Accordingly, the present invention provides a composition comprising nicotine-containing parts of a species of the genus Nicotiana, optionally in combination with one or more of the species Allium sativum, Zingiber officinale and Curcuma longa; wherein the composition is in the form of a liquid nasal, liquid oral, or liquid ocular formulation.

Preferably, the composition also comprises a pharmaceutically acceptable carrier.

A powdered composition of tobacco as a nasal formulation is known; and referred to as snuff or rapé. Such a nasal formulation, per se, is not included within this invention. However, there is no prior suggestion that tobacco for liquid nasal administration is useful as an antiviral agent, nor that the therapeutic value of the formulation may be enhanced by combination with other plant materials.

Thus, one embodiment of the present invention provides a composition comprising nicotine-containing parts of a Nicotiana species as the sole plant species.

In a further embodiment, the present invention provides a composition comprising nicotine-containing parts of a Nicotiana species in combination with one or more of the species Allium sativum, Zingiber officinale and Curcuma longa.

Suitable liquid nasal formulations of this invention include nasal drops, nasal sprays, nasal nebulizers, aerosols, and nasal inhalation solutions.

Suitable liquid oral formulations of this invention include oral drops, oral sprays, oral nebulizers, aerosols, oral inhalation solutions, liquid capsules, lozenges, liquids, syrups, elixirs, and suspensions.

Suitable liquid ocular formulations of this invention include eye drops.

Preferred forms of the compositions of this invention are nasal drops, nasal sprays, nasal nebulizers, nasal inhalation solutions, oral inhalation solutions for oral inhalers, oral drops, aerosols, and eye drops

Preferred formulations are a nasal spray or oral drops, especially a nasal spray.

For example, the compositions of this invention may be administered in an oral or nasal inhaler, a nasal spray, a pressurized aerosol, nasal drops, or eye drops. The composition may be nasally administered with a nasal spray device to deliver a mist spray to nasal mucosa, for example a spray-squeeze bottle or a pump vaporizer

Nicotiana is a genus of herbaceous plants, commonly known as tobacco plants. The subgenera of the Nicotiana genus include tabacum, rustica, and petunioides. All the species of subgenera may be used in the compositions of this invention including, for example, N. acuminate, N. Africana, N. alata, N. attenuata, N. benthamiana, N. clevelandii, N. glauca, N. glutinosa, N. langsdorffii, N. longiflora, N. occidentalis, N. obtusifolia, N. otophora, N. Plumbaginifolia, N. quadrivalvis, N. rustica L, N. suaveolens, N. sylvestris, N. tabacum L, N. tomentosiformis.

The most widely used species is N. tabacum L, which is grown worldwide for production of tobacco products including cigarettes.

Preferred species of tobacco are Nicotiana tabacum L, Nicotiana rustica L and Nicotiana sylvestris.

The parts of the tobacco species used in the present compositions are those that contain nicotine. This includes, for example the leaves, stems, roots and flowers of Nicotiana species. The seeds of tobacco do not contain nicotine. Preferable the compositions of this invention contain leaves of Nicotiana species.

The compounds present in tobacco include: nicotine; nornicotine; anabasine; anatabine; cotinine; anabaseine; myosmine (3-(3,4-Dihydro-2H-pyrrol-5-yl) pyridine); N-formylnornicotine; isonicotine [4-(4-piperidinyl)pyridine]; and nicotyrine [3-(1-methylpyrrol-2-yl) pyridine].

The main constituent of the composition of this invention is one or more species of the genus Nicotiana. Nevertheless, the composition and administration routes of this invention do not have the toxicity and addictive properties associated with tobacco products, since this composition does not have toxic and/or addictive additives, usually present in cigarettes. Furthermore, because the compositions of this invention provide and antiviral medicinal product, there is no social behaviour component linked to psychological aspects of addiction.

Although smoking tobacco is generally regarded to be carcinogenic, in fact the toxic effects caused by cigarette smoking are not primarily due to nicotine, but instead the carcinogens and other toxins produced by the burning and inhalation of tobacco. Nicotine itself is not carcinogenic. Other known routes of administration of nicotine, free from and chemical additives that are not present in the tobacco plant, have proven therapeutic effects in multiple pathologies.

Furthermore, the degree of addiction of natural nicotine can be equated to that of caffeine. Wild tobacco itself does not contain toxins that induce or enhance addictive effects, which are present in industrial cigarettes.

In addition to a Nicotiana species, the compositions of this invention may contain one or more the species Allium sativum, Zingiber officinale and Curcuma longa and Piper. Each constituent acts in synergy with the Nicotiana species to enhance its effect as an adjuvant or modulate its activity against Covid-19 complications such as cardiovascular damage, neurocognitive disorders, or neuronal degeneration.

Allium sativum is commonly known as garlic. Garlic is well known for human consumption and use, especially as a common seasoning worldwide. The compounds present in garlic include S-allylcysteine (SAC), S-allyl mercaptocysteine (SAMC) and diallyl disulfide. These compounds act in synergy to protect tissues against oxidative stress. Preferably, the form of garlic used contains SAC and/or SAMC.

Zingiber officinale is a flowering plant whose rhizome, ginger, is widely used as a spice and a folk medicine. The compounds present in ginger include 6-shogaol, 6-gingerol, 8-gingerol and 10-gingerol. These compounds act in synergy to diminish the production of inflammatory cytokines observed in severe and critical Covid-19 patients; support enterocites regulation; and provide neuroprotection.

Curcuma longa, known commonly as turmeric, is a flowering plant of the ginger family, Zingiberaceae, the roots of which are used in cooking. The compounds present in turmeric include curcumin, demethoxycurcumin, and bisdemethoxycurcumin. Those compounds act in synergy to effect the metabolic inactivation of the amino acid sequence used by SARS-COV-2 S glycoprotein in its binding to ACE2 human enzyme. Those compounds also exhibit properties in regulating the anti-inflammatory cholinergic pathway disrupted in Covid-19 disease.

One suitable composition of this invention comprises nicotine-containing parts of a Nicotiana species in combination with Allium sativum, Zingiber officinale, and Curcuma longa.

A preferred composition of this invention comprises a combination of Nicotiana, Allium sativum, and Zingiber officinale.

The solid constituents of the compositions of this invention may comprise 100% Nicotiana nicotine-containing parts. If other plant constituents are present in the compositions, the proportion of Nicotiana leaves may be suitably from 50% to 90% by weight of the constituents. Preferably Nicotiana leaves comprises from 70% to 90%, for example from 80 to 90% by weight of the constituents.

If Allium sativum (garlic) is present in the constituents, it may comprise from 1% to 30%, suitably from 5% to 20%, preferably from 10% to 15%, for example from 11 to 14% by weight of the constituents.

If either Zingiber officinale (ginger) or Curcuma longa (turmeric) is present in the constituents, each may comprise from 0.1% to 10%, suitably from 0.5% to 2%, preferably from 1% to 1.5%, for example from 1.1 to 1.4% by weight of the constituents.

In one embodiment, the present invention provides a composition in the form of oral drops which composition comprises Nicotiana, Allium sativum, Zingiber officinale and Curcuma longa.

A preferred embodiment of this invention provides a nasal spray comprising Nicotiana, Allium sativum, Zingiber officinale, and Curcuma longa.

The compositions of the invention may further comprise a pharmaceutically acceptable carrier. The carrier may include one or more excipients or diluents, such as solvents, dispersion media, coatings, isotonic and absorption delaying agents, binders, lubricants, disintegrants, colouring agents, bulking agents, flavouring agents, sweetening agents, and buffers. Examples of such excipients include gelatin, gum arabicum, lactose, microcrystalline cellulose, starch, sodium starch glycolate, calcium hydrogen phosphate, magnesium stearate, talcum, and colloidal silicon dioxide. Suitable buffers include chloride, citrate, acetate, borate, formate, and phosphate buffers. Preferred buffers are citrate buffers.

The pharmaceutically acceptable carrier may be a liquid. Liquid carriers are used in preparing solutions, suspensions, emulsions, syrups, elixirs, drops, sprays, and pressurized compositions. Examples of liquid carriers are water, an organic solvent, a mixture of both, or a pharmaceutically acceptable oil or fat.

A preferred excipient for oral, nasal, or ocular formulations is sterile water, optionally with an alcohol, such as ethanol.

The compositions of this invention may also contain a preservative and/or stabilizer. These include, for example: ethylene diamine tetra-acetic acid, lower alkyl p-hydroxybenzoates, chlorhexidine, quaternary ammonium compounds, for example cetylpyridinium chloride, tetradecyltrimethyl ammonium bromide, and myristyl picolinium chloride. Each of these compounds may be used in a concentration of 0.002 to 0.05%, for example 0.02% (weight/volume in liquid compositions, weight/weight in solid compositions).

The compositions of this invention may also be formulated by encapsulation in a colloidal drug delivery system. Suitable colloidal drug delivery systems include lipid-based and polymer-based colloidal drug delivery systems such as liposomes, nanoparticles, microparticles or block copolymer micelles.

Alternatively, the compositions may be encapsulated in a polymeric, non-colloidal drug delivery system, such as hydrogels and films.

The compositions of the invention may also be formulated as nanoparticles, for example with a mean particle size of 0.13 μm. The mean particle size of normal powder is 22.87 μm.

The compositions of the invention are prepared by mixing appropriate quantities of extracts of nicotine-containing parts of Nicotiana, preferably the leaves, with the other constituents; the mixture may be ground using a wet grinding mill. The constituents of the composition may be in the form of the raw plant material, or my be prepared from extracts of the relevant plant materials. Extracts of Nicotiana leaves may be prepared by finely cutting tobacco leaf, preferably at the beginning of the aging phase, extracting in water or an aqueous alcoholic solvent, macerated and then filtered. Extracts of Allium sativum may be prepared by freshly peeling garlic, crushing, blending in water or an aqueous alcoholic solvent, macerated and then filtered. Extracts of Zingiber officinale may be prepared by freshly peeling ginger rhizome, crushing, blending in an aqueous alcoholic solvent, macerated and then filtered. Extracts of Curcuma longa may be prepared by freshly peeling turmeric rhizome, crushing, blending in an aqueous alcoholic solvent, macerated and then filtered.

In the nasal spray or eye drop forms of the composition of the present invention, suitably, 0.005 to 1.3 ml of the liquid composition may be released per individual application, preferably from 0.01 to 0.25 ml per application, for example from 0.05 to 0.25 ml per application. The amount of Nicotiana delivered from a nasal spray or eye drop formulation per application may be suitably from 1.2 mg to 325 mg, preferably from 2.5 mg to 62 mg, for example from 12 to 62 mg of Nicotiana.

The compositions of this invention can be administered to a mammal, including human, for the prevention or treatment of a viral disease caused by a coronavirus, for example a viral disease caused by a betacoronavirus, in particular a viral disease caused by the coronavirus SARS-CoV-2, in particular Covid-19.

The compositions are particularly useful for human treatment.

Betacoronavirus (β-CoVs or Beta-CoVs) is one of four genera (Alpha-, Beta-, Gamma-, and Delta-) of coronaviruses. Member viruses are enveloped, positive-strand RNA viruses that infect mammals (of which humans are part). The natural reservoir for betacoronaviruses are bats and rodents. Rodents are the reservoir for the subgenus Embecovirus, while bats are the reservoir for the other subgenera.

The coronavirus genera are each composed of varying viral lineages with the betacoronavirus genus containing four such lineages: A, B, C, D. This genus has previously been also known as “group 2 coronaviruses”. The genus is in the subfamily Orthocoronavirinae in the family Coronaviridae, of the order Nidovirales.

The betacoronaviruses of the greatest clinical importance concerning humans are OC43 and HKU1 (which can cause the common cold) of lineage A, SARS-CoV and SARS-CoV-2 (which causes the disease COVID-19) of lineage B, and MERS-CoV of lineage C. MERS-CoV is the first betacoronavirus belonging to lineage C that is known to infect humans.

Within the genus Betacoronavirus (Group 2 CoV), four subgenera or lineages (A, B, C, and D) have traditionally been recognized. The four lineages have also been named using Greek letters or numerically. A fifth subgenus, Hibecovirus, was added more recently. Member subgenera and species include:
Embecovirus (lineage A), including: Betacoronavirus 1; Bovine coronavirus; Human coronavirus OC43; China Rattus coronavirus HKU24; Human coronavirus HKU1; Murine coronavirus; Mouse hepatitis virus; and Myodes coronavirus 2JL14
Sarbecovirus (lineage B), including: Severe acute respiratory syndrome-related coronavirus (SARSr-CoV or SARS-CoV); Severe acute respiratory syndrome coronavirus (SARS-CoV or SARS-CoV-1); Severe acute respiratory syndrome coronavirus 2 (SARS-CoV-2); Bat SARS-like coronavirus WIV1 (Bat SL-CoV-WIV1); and Bat coronavirus RaTG13.
Merbecovirus (lineage C), including: Hedgehog coronavirus 1; Middle East respiratory syndrome-related coronavirus (MERS-CoV); Pipistrellus bat coronavirus HKU5; and Tylonycteris bat coronavirus HKU4
Nobecovirus (lineage D), including: idolon bat coronavirus C704; Rousettus bat coronavirus GCCDC1; Rousettus bat coronavirus HKU9
Hibecovirus, including: Bat Hp-betacoronavirus Zhejiang2013

Thus, the viral disease includes diseases within the coronaviruses family, such as, for example, SARS-Cov-1 and the common cold.

In addition, the viral disease may be any mutation or alternative strain of the SARS-CoV-2 virus.

The compositions of this invention are not only antiviral agents, but also anti-inflammatory and immunomodulatory agents.

Although the invention is not limited to a particular mechanism, it is believed that the compositions exhibit a direct antiviral action both by antagonizing the binding of the ACE-2 Spike protein and also by antagonizing the disruption of the nicotinic acetylcholine receptor (nAChR). Such a combination of antiviral actions means that the compositions act both in a protective, i.e. prophylactic, manner, as well as a direct therapeutic manner.

Furthermore, the compositions of the invention protect and regulate the Renin-Angiotensin-Aldosterone System (RAAS), which regulates kidney, lung, heart, intestinal and brain functions.

The unique combination of activities results in the compositions of this invention being useful for treating a further related condition. Persistent chronic fatigue symptoms have been frequently observed in survivors of viral infections, including Severe Acute Respiratory Syndrome (SARS). This has been observed in the case of SARS-Cov-1, in 2003, and more recently in SARS-CoV-2. For example, in COVID-19 infected patients that have overcome the disease, structural viral damage of organs may remain for a prolonged time. Multiorgan symptoms following an infection are being reported by increasing numbers of patients; the condition is known as Post-Covid-19 syndrome or ‘Long-Covid’. The symptoms include cough, breathlessness, chronic fatigue, headache, chest pain, joint pain, insomnia, anxiety and stress. It has been found that the compositions of this invention speed the recovery of patients suffering from such symptoms.

The compositions of this invention can therefore be administered to a mammal, including human, for the prevention or treatment of symptoms following a viral infection, in particular a SARS infection, such as Post-Covid-19 syndrome.

The dosages of the compositions of this invention are adjusted according to the form of administration. For example, a nasal spray formulation is more effective than an oral liquid formulation, so that smaller dose of the constituents by weight is required when applied via a nasal spray than via oral liquids.

If the administration route is the oral liquid form of the compositions of this invention, the dose of the plant constituents of this invention may be suitably from 20 mg to 4 g per day, preferably from 100 mg to 1 g per day, for example from 400 mg to 500 mg per day. The total daily dosage is conveniently administered in three portions of from 0.1 ml to 16 ml, preferably from 0.4 ml to 4 ml, for example from 1.6 ml to 2 ml of the oral liquid formulation.

If the administration route is the nasal spray or eye drops form of the compositions of this invention, the dose of the plant constituents of this invention may be suitably from 2.4 mg to 650 mg per day, preferably from 5 mg to 124 mg per day, for example from 24 mg to 124 mg per day. For a nasal spray, the total daily dosage is conveniently administered in a single spray application in each nostril per day. For eye drops, the total daily dosage is conveniently administered in single eye drops application in each eye per day, depending on the type of eye drop dispenser.

The dosages stated above are suitable for non-smoking adults as a prophylactic, or for health personnel at risk of contagion, or for patients with mild symptoms of the virus including SARS-CoV-2.

For patients with moderate to severe symptoms, especially if hospitalized, the daily dosage of an oral liquid formulation could be increased by 10% to 20% from the figures for prophylactic or mild symptoms; and the daily dosage of nasal spray could be doubled, suitably by administering two actuations per nostril per day, instead of one; and the daily dosage of eye drops spray could be doubled, suitably by administering two applications per eye per day, instead of one.

For patients with severe symptoms, especially those requiring intubation, the daily dosage of an oral liquid formulation could be increased by 30% to 40% from the figures for prophylactic or mild symptoms; the daily dosage of nasal spray could be trebled, suitably by administering three actuations per nostril per day, instead of one; and the daily dosage of eye drops could be trebled, suitably by administering three applications per eye per day, instead of one.

For non-smoking patients, it is also advantageous to administer eye drops or an oral liquid form prior to the use of a nasal spray. This initial application of a tobacco formulation prepares the body for the administration of a rapidly absorbed amount of Nicotiana, and so prevents any adverse reaction thereto.

For patients who smoke, the dosages of an oral liquid formulation may be increased by 20% to 70%; and the dosage of a nasal formulation could be up to doubled, depending on the level of smoking. Because females have lower serum ACE2 levels than males, the dosages for females may be 70% of the dosages for males.

The present invention is illustrated by the following examples.

### EXAMPLE 1

### Oral Drops

Oral drops were prepared by from extracts of plant ingredients, by dissolving in 28% aqueous ethanol in the following amounts:
204 mg/ml Nicotiana tabacum L leaves (85% by weight)
30 mg/ml garlic (12.5% by weight)
3 mg/ml Curcuma (1.25% by weight)
3 mg/ml ginger (1.25% by weight)

The resulting oral drop solution contained a total of 240 mg/ml of the plant ingredients.

### EXAMPLE 2

### Liquid Spray with Tobacco

An extract of the tobacco species Nicotiana tabacum L leaves was dissolved in distilled water containing 0.5% phenylethyl alcohol in the proportion of 0.25 g. of tobacco per ml of solution.

This solution may be used as a nasal spray, or an oral inhalation spray.

### EXAMPLE 3

### Liquid Spray with Tobacco and Garlic

Extracts of garlic and leaves of the tobacco species Nicotiana rustica were dissolved in distilled water containing 0.5% phenylethyl alcohol in the following proportions:
212 mg/ml Nicotiana rustica (87.63%)
30 mg/ml garlic (12.37%)

This solution may be used as a nasal spray, or an oral inhalation spray.

### EXAMPLE 4

### Liquid Spray with Tobacco, Garlic, and Ginger

Extracts of garlic, ginger, and leaves of the tobacco species Nicotiana tabacum L were dissolved in distilled water containing 0.5% phenylethyl alcohol in the following proportions:
208 mg/ml Nicotiana tabacum L (86.34%)
30 mg/ml garlic (12.40%)
3 mg/ml ginger (1.24%)

This solution may be used as a nasal spray, or an oral inhalation spray.

### EXAMPLE 5

### Clinical Evidence

The therapeutic action of the composition of Example 4 (“Composition A”) was assessed on the symptoms in a group of human patients infected with SARS-CoV-2 versus a control group.

### Methods

Randomized open-label trial pilot study where 80 patients were randomly assigned to receive standard of care (SOC) treatment as outpatient treatment after PCR+test; SOC plus Composition A was administered in the intervention group (40 patients). SOC was administered in the control group (40 patients).

Basic Odds Ratio study (95% confidence interval) in 40 patients for intervention group and 40 patients for the control group. The evaluation in the groups was carried out during 15 days assessing the improvement or worsening of each symptom daily.

An Odds Ratio is a relative measure of effect, which allows the comparison of the intervention group relative to the control group. The numerator is the odds in the intervention arm. The denominator is the odds in the control arm=Odds Ratio (OR). Thus, if the OR is <1 the intervention is better than the control

### Results

| Symptom | Odds Ratio |
|---|---|
| Fever | 0.897 |
| Cough | 0.571 |
| Dyspnea | 0.460 |
| Muscle fatigue | 0.250 |
| Cephalea | 0.570 |
| Ageusia | 0.150 |
| Anosmia | 0.650 |
| General malaise | 0.316 |
| Nasal congestion | 0.890 |

It can be seen that for every symptom, the Odds Ratio value is less than 1, demonstrating a significant improvement of these symptoms in the group given Composition A, compared to the control group.

1. A composition comprising nicotine-containing parts of a species of the genus Nicotiana, either as the sole plant species or in combination with one or more of the species Allium sativum, Zingiber officinale and Curcuma longa; wherein the composition is in the form of a liquid nasal, liquid oral, or liquid ocular formulation.

2. The composition as claimed in claim 1, comprising nicotine-containing parts of a Nicotiana species as the sole plant species.

3. The composition as claimed in claim 1, comprising nicotine-containing parts of a Nicotiana species in combination with one or more of the species Allium sativum, Zingiber officinale and Curcuma longa.

4. The composition as claimed in claim 3, which comprises a combination of Nicotiana, Allium sativum, Zingiber officinale and Curcuma longa.

5. The composition as claimed in claim 3, which comprises a combination of Nicotiana, Allium sativum, and Zingiber officinale

6. The composition as claimed in claim 3, wherein Nicotiana comprises from 80 to 90% by weight of the constituents.

7. The composition as claimed in claim 1 in the form of oral drops, or nasal spray.

8. The composition as claimed in claim 7 in the form of a nasal spray.

9. The composition as claimed in claim 7 in the form of oral drops, which composition comprises a Nicotiana species, Allium sativum, Zingiber officinale and Curcuma longa.

10. The composition as claimed in claim 8 in the form of a nasal spray, which composition comprises a Nicotiana species, Allium sativum, Zingiber officinale, and Curcuma longa.

11. A method of prevention or treatment of a viral infection in mammals, including humans, which comprises administering to a mammal a composition comprising nicotine-containing parts of a species of the genus Nicotiana, either as the sole plant species or in combination with one or more of the species Allium sativum, Zingiber officinale and Curcuma longa; wherein the composition is administered in the form of a liquid nasal, liquid oral, or liquid ocular formulation.

12. The method as claimed in claim 13 11 for the prevention or treatment of a disease caused by the coronavirus SARS-CoV-2.

13. The method as claimed in claim 11 for the prevention or treatment of Covid-19.

14. The method as claimed in claim 11 wherein the composition is administered in the form of a nasal spray.

15. The method as claimed in claim 11 wherein the composition comprises nicotine-containing parts of a Nicotiana species as the sole plant species.

16. (canceled)

17. The method of claim 11 wherein the viral infection is a disease caused by a beta coronavirus.

18. (canceled)

19. (canceled)
